(19) United States
(12) Patent Application Publication
(10) Pub. No.: US 2015/0112326 A1
(43) Pub. Date: Apr. 23, 2015

(54) METHOD TO REDUCE SENSE OF TASTE AND FOOD INTAKE

(71) Applicant: Hong Li, Jilin City (CN)

(72) Inventor: Hong Li, Jilin City (CN)

(21) Appl. No.: 14/058,202

(22) Filed: Oct. 18, 2013

(51) Int. Cl. A61B 18/00

(52) U.S. Cl. CPC A61B 18/00
USPC 606/41

(57) ABSTRACT

A method for reducing sense of taste and food intake is by ablating the mouth tissue that affecting sense of taste, selected from the group comprising: salivary glands, nerves and blood vessels that affect salivary glands' function, salivary channels, salivary exit ports, tongue papillae, tongue mucosa, tongue nerves, tongue arteries and veins. The ablation system comprises: an energy source and a catheter.

### RELATED APPLICATION

This application is entitled to the benefit of Provisional Patent Application Number 61/729,428, filed 23 Nov. 2013.

### BACKGROUND OF THE INVENTION

1. Field of the Invention

This invention is in the field of minimally invasive treatment of patients, by using medical devices, especially using ablation devices to treat overweight and obese.

2. Background Information

Overweight (and obese) is a rising epidemic, especially in developed countries. It affects nearly 200 million Americans, and 300 million Chinese. There are more than 1.5 billion people worldwide overweight.

Overweight (and obese) has been causing many health problems such as diabetes, hypertension, heart decease and so on. It greatly increases medical care cost, and reduces productivity.

Many efforts have been made worldwide to reduce or prevent overweight (and obese), such as by drugs, surgery intervention in GI track with medical devices, exercise, and dieting. But there is still a very large population of overweight, indicating the existing methods are often not very effective, or not safe to use such as some drugs.

The causes of overweight (and obese) are: less and less physical activities, more and more tasty food available. Since it is very hard to have more physical activities, and to limit food supply, it is nature to look into the ways to easily control food intake for weight control.

It is an object of the present invention to provide a method and a system to reduce sense of taste for overweight people, to eat less, and to reduce body weight over the time, by ablating some tissues inside the mouth.

In U.S. Pat. No. 5,707,349, ablation of tongue tissue without ablating the top surface mucosa was described to reduce volume of the tongue for opening up airway, especially at the area near the end of the tongue. To avoid ablating the mucosa at the top surface of the tongue, needle electrodes were used to penetrate into the tongue, and also the mucosa was cooled by a fluid during ablation. In U.S. Pat. No. 6,409,720, concaved ultrasound transducers were used to focus the ablation energy into the tissue below the mucosa. Similar apparatus can also be used to ablate some areas of the tongue in the method mentioned in the present invention.

### BRIEF SUMMARY OF THE INVENTION

In the present invention, a method of reducing sense of taste to reduce food intake for weight loss is by ablating at least one selected from the group comprising: salivary glands, nerves and blood vessels that affect salivary glands' function, salivary channels, salivary exit ports, tongue papillae, tongue mucosa, tongue nerves, tongue arteries and veins.

### BRIEF DESCRIPTION OF THE SEVERAL VIEWS OF THE DRAWINGS

FIG. 1 is a schematic view of the ablation system of the present invention.

FIG. 2 is a schematic view of the ablation catheter with four ablation heads with their tips in one plan and spaced apart from each other.

### DETAILED DESCRIPTION OF THE INVENTION

It is well known that ablating tissue at temperature between 50 to 90° C. or below −50° C. in less than a minute can cause cells to necrosis and to loss their normal functions.

In the present invention, a method of ablating different structures and areas of the mouth tissue is described to reduce mouth's function of sense and taste to food by different mechanisms to different extends.

1. Ablating salivary glands can reduce saliva, which can reduce sense of taste. Over weighted and obese people often have excessive saliva produced especially when eating, which makes them eat more than necessary.

Only some portions of salivary glands will be ablated so that some quantity of saliva is still produced by un-ablated portions of salivary glands to maintain normal sense of taste.

There are three types of large salivary glands: parotid gland, submandibular gland, and sublingual gland. And there are some small salivary glands. They are located at different areas in the mouth.

2. Ablating salivary channels and exit ports can make them less elastic, and therefore can reduce saliva flow through them. When ablation temperature is high and close to 90° C., saliva channel and exit port can be partially or completely sealed by thermo fusion at the ablation site when pressed during ablation. That can reduce saliva flow into the mouth cavity.

3. Ablating mouth blood vessels including arteries and veins to make them less elastic, and therefore to have reduced flow of blood to the tissues such as salivary glands that affect sense of taste, so that sense of taste will be reduced.

4. Ablating nerves in the mouth area that affect sense of taste directly such as nerves at the tongue's tip area, or indirectly such as the nerves connected to salivary glands, to reduce sense of taste.

Ablation on nerves especially tongue's nerves should be mild and minimum so that normal sense, taste and movement of the mouth tissue will be maintained.

5. Ablating the top surface layer structures of the tongue, such as tongue papillae, tunica mucosa linguae, foramen caecum linguae, especially at the tip area of the tongue and nearby, where sense of taste is strong. Tongue papillae's function is sense of taste.

Ablation areas on tongue's top surface layer can be made non-continuous, so that the areas of normal un-ablated healthy tissue remain in between the ablated areas to maintain some level of sense of taste to eat necessary quantity of food for health.

Ablation on the mouth surface tissue should be mild and minimum, so that the surface (especially tongue mucosa) will not be infected or inflammated.

6. Ablating the bottom layer structures of the tongue, such as GI.apicis linguae to reduce saliva passage, arteries and veins to make them less elastic and less efficient for blood flow, so that tongue's function of sense of taste will be reduced. Tongue's main arteries and veins are concentrated at the bottom layer of the tongue.

7. Ablating subsurface and/or middle layer structures of the mouth especially the tongue, without ablating the outer surface to avoid the outer surface being inflammated and infected. Ablating the subsurface structures of the tongue such as vessels of blood and fluid, can reduce sense of taste.

8. Ablating side walls of the mouth, to ablate parotid gland and salivary channels.

9. Ablating the tissue below the tongue, to ablate submandibular gland, sublingual gland, salivary channels and salivary exit ports.

For the mouth surfaces and the tongue's nerves, ablation can be minimized by shorter ablation duration, and by lower temperature (above 50° C., below 60° C.) for heat-induced ablation, and higher temperature for cryoablation. In other areas, normal heat ablation temperature can be around 70° C. Ablation temperature close to 100° C. can char the tissue, should be avoided.

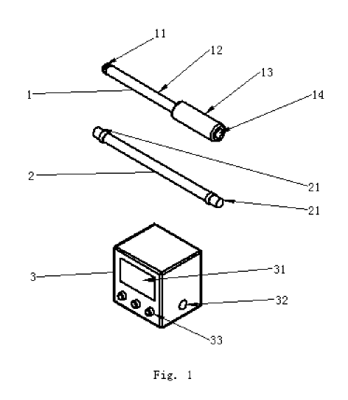

In the present invention as shown in FIG. 1, a system of ablating the targeted mouth tissue comprises an ablation catheter 1, a controller 3, and a connection cable 2 to connect the catheter and the controller.

The ablation catheter 1 has an ablation head 11 at the distal end to transmit ablation energy to the mouth tissue, a handle 13 at the proximal end to hold and manipulate the head, and a shaft 12 to connect together the head and the handle. The catheter length is 15 to 30 cm, similar to a tooth brush length, to easily manipulate and apply pressure onto the head for good tissue contact during ablation. A temperature sensor such as a thermocouple can be attached to the ablation head at the side close to the tissue to be ablated. At the proximal end of the handle, there is a connection port 14 for one connector 21 of the connection cable 2 to plug in. The other connector 21 of the connection cable can be plugged into connection port 32 of the controller 3.

Catheter with only one ablation head 11 in FIG. 1 is good to reach small area and corner which are difficult to reach such as the tissue blow the tongue. To make two ablations in one line quickly with a controlled distance, a ring shape head can be put proximal to the distal head 11 with a distance.

To ablate a large area quickly with controlled distance(s) between the ablated areas, more ablation heads can be installed at the distal section of the catheter.

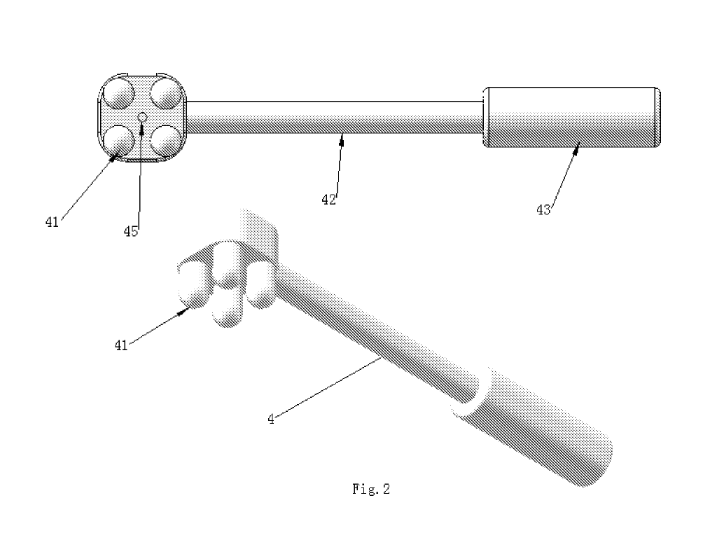

In FIG. 2, there are four ablation heads 41, which are spaced apart so that the tissue between the heads is not in contact with the ablation heads and is not ablated during ablation procedure, to maintain normal function of sense, taste, supply of blood and fluid, and muscle movement. The tips of the heads are in one plan, which are good for ablating large flat tissue surface such as the top surface of the tongue, and the side wall surfaces of the mouth near parotid gland and cheek. In FIG. 2, 42 is catheter shaft, 43 is catheter handle, and 45 is fluid exit hole to cool the heads and the tissue nearby.

The ablation controller 3 comprises an energy source, an automatic control to deliver energy according to parameters preset and sensed, a touch screen display/control panel 31 to preset and display ablation parameters such as ablation temperature, power and time. Ablation parameters can be preset by adjusting nubs 33, or buttons on the panel. On the controller 3, there is a connection port 32 for one connector 21 of the connection cable 2 to plug in.

Ablation energy can be radio frequency current, electromagnetic, ultrasound, microwave, laser, infrared light, hot water, and cryogenic cooling. When laser or infrared light is used as ablation energy source, the ablation head can be made with an optically transparent material. For non-optical energy sources, the ablation head can be made with a metallic material, or a thin plastic balloon filled with fluid to conduct heating or cooling energy.

The ablation controller 3 can be made very simple in some cases. For example, for cryoablation of the mouth tissue, the ablation controller can be just a hand size bottle with compressed cryogenic coolant in it. To start ablation, just connect the cryogenic bottle directly onto the ablation catheter with its ablation head already in contact with the tissue to be ablated. Then the cryogenic coolant will be delivered into the ablation head and start cooling, all by expansion of the coolant. The ablation temperature is controlled by the size of the bottle, the type and the quantity of the coolant, and the design of the catheter. This type of simple ablation devices was already used in cardiac application.

To ablate the subsurface tissue without ablating the surface tissue of the mouth, a room temperature fluid such as saline can be delivered to the ablation site and/or the ablation head during a heat induced ablation to cool the surface of the mouth tissue. There is a fluid cooling channel in the catheter, the connection cable and the controller; there is a fluid exit hole 45 near ablation heads 41 (FIG. 2); there is a fluid pump and a control circuit in the controller, to cool the ablation head during ablation. During a heat induced ablation with fluid cooling, ablation head and the tissue surface in contact with the head will be cooled to a temperature range below 40° C., so that the tissue surface will not be ablated. But the subsurface tissue will be ablated by the energy delivered into it from an energy source such as ultrasound, microwave, and radiofrequency current. In such a case, ablation is controlled by a preset power, and safeguarded by the head temperature to not exceed 40° C.

Transducers with concaved front surface can also be used to generate ultrasound energy and focus it into a certain distance, and ablate the tissue below the tissue surface in contact with or near the head, without ablating the surface tissue.

To ablate deep subsurface tissue, a metallic needle ablation head can also be used. The needle head can be pushed into the mouth tissue and then deliver ablation energy such as radio frequency current. The areas with artery and vein of the mouth should not be pierced by the needle.

The fluid cooled ablation, the concaved transducers, and the needle ablation heads could also be used to do ablation from outside of the mouth, such as from facial skin near cheek to ablate parotid gland and other mouth tissue without ablating skin.

Ablation effects could decay over the time, as some of the ablated tissue could gradually heal. When that occurs, patients can be re-ablated to get desired effects. When desired, some degree recovery could be good to get some appetite back. Healing of some ablated tissue to some extend is depending on the injury level during ablation. For heat induced ablation, the higher the ablation temperature, the more severe the damage. For cryoablation, the lower the ablation temperature, the more severe the damage.

Ablation on the mouth tissue can be done in a doctor's office with local anesthesia. It is a simple and quick procedure. It can be done a few times. So ablation dose can be increased gradually to achieve a desired result without over dosing. That is to ablate mildly in small areas first, and have the patient come back and get ablated more according to the follow up result, so that the patient will not lose too much appetite and body weight.

Ablation sites will be easily checked by a patient and a doctor during follow up after ablation. And if there is inflammation or infection at the ablation sites, it can be easily treated.

Here is an example of the ablation procedure:

1. Preset ablation parameters such ablation temperature, time, and power from the display/control panel on the ablation controller.

2. Placing at least one ablation head into the mouth at a desired location. Ablation head should be in good contact with the tissue to be ablated, when ablation energy is radio frequency current, or ultrasound, microwave, hot water, or cryogenic cooling. Good contact between the ablation head and the tissue to be ablated can be achieved by pressing the ablation head onto the tissue with the catheter handle.

3. Turn on the ablation power and supply the ablation energy onto the ablation head, and start to ablate the mouth tissue with the preset parameters.

4. Place the ablation head onto another location, and start another ablation. Remove the ablation head out of the patient's mouth when the ablation procedure is completed.

I claim:

1. A method for reducing sense of taste and body weight is by ablating targeted mouth tissue that affects sense of taste; said tissue being selected from the group comprising: salivary glands, nerves and blood vessels that affect glands' function, salivary channels and salivary exit ports, tongue papillae, tongue mucosa, tongue nerves, tongue arteries and veins, using an ablation system comprising an energy source and a catheter; said ablation energy source being selected from the group comprising electromagnetic, radio frequency, ultrasound, microwave, laser, infrared light, hot water, and cryogenic cooling; said catheter having at least one ablation head for transmitting ablation energy to the tissue to be ablated.

2. The ablation system of claim 1 wherein said ablation catheter has at least two ablation heads with a distance apart from each other for non-continuous ablations.

3. The ablation system of claim 1 wherein said ablation catheter has at least four ablation heads with their tips in one plan and spaced apart from each other for non-continuous ablations.

4. The ablation system of claim 1 wherein said ablation head has a temperature sensor attached to it for sensing ablation temperature.

5. The ablation system of claim 1 wherein said ablation head is a metallic needle.

6. The ablation system of claim 1 wherein said ablation head is a thin plastic balloon.

7. The ablation system of claim 1 wherein said ultrasound energy is generated by a transducer with a concaved front surface to focus the energy and ablate the tissue below the tissue surface at a distance from said head.

8. The ablation system of claim 1 wherein the length range of said catheter is 15 to 30 cm.

9. The ablation system of claim 1 further comprises a controller to preset, automatic control, and display ablation parameters; said ablation parameters being selected from the group comprising: temperature, time, and power.

10. The ablation system of claim 1 has at least one fluid cooling channel in said catheter, and a fluid pump, to cool said ablation head during ablation.

11. A method for reducing sense of taste and food intake is by ablating the mouth tissue selected from the group comprising: the tongue tissue, the tissue below the tongue, the tissue at side wall of the mouth.

12. The method of claim 11 wherein said tissue is the top surface tissue of the tongue.

13. The method of claim 12 wherein said top surface tissue of the tongue is selected from the group comprising: tongue papillae, and tongue mucosa.

14. The method of claim 12 wherein said top surface tissue of the tongue is to be ablated at a temperature range above 50° C. and below 60° C.

15. The method of claim 11 wherein said tissue is the bottom surface tissue of the tongue.

16. The method of claim 11 wherein said tissue is the middle layer tissue of the tongue.

17. The method of claim 11 wherein said tissue is the subsurface tissue of the mouth.

18. A method for reducing sense of taste and food intake is by ablating at least one type of the mouth tissue selected from the group comprising: salivary glands, nerves and blood vessels that affect glands' function, salivary channels and salivary exit ports.
